(19) United States
(12) Patent Application Publication
(10) Pub. No.: US 2010/0135854 A1
(43) Pub. Date: Jun. 3, 2010

(54) BIOSENSOR HAVING TRANSISTOR STRUCTURE AND METHOD OF FABRICATING THE SAME

(75) Inventors: Yong Suk YANG, Daejeon (KR); Seong Hyun Kim, Daejeon (KR); Sang Chul Kim, Daejeon (KR); Doo Hyeb Youn, Daejeon (KR); Zin Sig Kim, Daejeon (KR)

Correspondence Address: RABIN & Berdo, PC, 1101 14TH STREET, NW, SUITE 500, WASHINGTON, DC, 20005, US

(73) Assignee: ELECTRONICS AND TELECOMMUNICATIONS RESEARCH INSTITUTE, Deajeon (KR)

(21) Appl. No.: 12/536,021

(22) Filed: Aug. 5, 2009

(30) Foreign Application Priority Data
Dec. 3, 2008 (KR) 10-2008-0121622
Mar. 13, 2009 (KR) 10-2009-0021592

(51) Int. Cl. G01N 33/53; B05D 5/12

(52) U.S. Cl. 422/68.1; 427/2.13

(57) ABSTRACT

Provided are a biosensor and a method of fabricating the same. The biosensor has a transistor structure including a gate electrode formed on a substrate, a gate insulating layer formed on the gate electrode, source and drain electrodes formed on the gate insulating layer, and a channel region formed between the source and drain electrodes. Here, the channel region includes an active layer formed of an active polymer sensing an antigen-antibody reaction and a hydrophilic nano particle. The active layer is formed through direct printing, for example, inkjet printing. The biosensor having such a structure can be increased in reactivity between an antigen and an antibody and hydrophilicity to improve the sensor's characteristics, fabricated in a large-area process using direct printing, and further facilitates formation of devices on various substrates formed of, for example, plastic.

### CROSS-REFERENCE TO RELATED APPLICATION

This application claims priority to and the benefit of Korean Patent Application Nos. 10-2008-0121622, filed Dec. 3, 2008 and 10-2009-0021592, filed Mar. 13, 2009, the disclosure of which is incorporated herein by reference in its entirety.

### BACKGROUND

1. Field of the Invention

The present invention relates to a biosensor sensor having a transistor structure and a method of fabricating the same, and more particularly, to a biosensor in which the sensor's performance such as sensitivity and selectivity is improved by forming an active layer using an active polymer capable of sensing an antigen-antibody reaction and a hydrophilic nano particle in a channel region of a transistor through direct printing, and a method of fabricating the same.

2. Discussion of Related Art

Generally, organic semiconductors are made by vacuum evaporation using heat or plasma, or a liquid phase process such as inkjet printing. With the development of science, these electronic materials are actually being applied to almost all fields, including aeronautics and space science, biotechnology, environment and energy technology, material industry, medicine and pharmacy, electronics and computers, and security and safety, one of which is transistor devices.

The organic transistor has source, drain and gate electrodes, and a channel region formed of an organic semiconductor.

The organic semiconductor forming the channel region of the transistor is a novel material, which has semiconductor or metal characteristics and high electric conductivity. In addition, the organic semiconductor is suitable for fabricating low-cost and large-sized electronic devices, since it is lightweight and formed in a simple process, compared to conventional Si semiconductor devices.

Research into developing novel application devices using an organic semiconductor is being conducted, as is a variety of research in medical and biotechnological fields.

Research into biosensors has so far mostly focused on diagnostic sensors using antigen-antibody reactions. The antigen-antibody reactions are very specific, and thus considered a very valuable tool in accurately diagnosing diseases. However, there is a disadvantage in that antibodies are composed of protein, which is not stable.

Therefore, there is a need to develop highly sensitive biosensors having substrate specificity that is similar to or higher than antibodies, which are composed of protein, and stability that is higher than antibodies.

### SUMMARY OF THE INVENTION

The present invention is directed to a biosensor having a transistor structure in which a channel region is formed using a hydrophilic nano particle and an active polymer sensing an antibody-antigen reaction to increase reactivity between an antigen and an antibody and hydrophilicity and improve the sensor's performance.

The present invention is also directed to a method of fabricating a biosensor having a transistor structure, including forming an active layer using an active polymer material and a hydrophilic nano particle in a channel region of a transistor through direct printing such as inkjet printing.

One aspect of the present invention provides a biosensor having a transistor structure including: a gate electrode formed on a substrate; a gate insulating layer formed on the gate electrode; source and drain electrodes formed on the gate insulating layer; and a channel region formed between the source and drain electrodes, wherein the channel region includes an active layer formed of an active polymer sensing an antigen-antibody reaction and a hydrophilic nano particle.

The active polymer sensing the antigen-antibody reaction may have a conductive main chain showing semiconductor characteristics, and a side chain substituted with an aptamer or label specifically binding to a target material. The conductive main chain of the active polymer may be poly(3-hexylthiophene) (P3HT), poly(9,9-dioctylfluorene-co-bithiophene) (F8T2) or poly(3,3′-didodecyl-quaterthiophene) (PQT-12), and the side chain may be biotin.

The hydrophilic nano particle may be a composition of a nano particle such as alumina (Al₂O₃), aluminum nitride (AlN), silicon oxide (SiO₂) or boron oxide (B₂O₃), a cation interfacial active agent and a hydrophilic polymer.

Here, the active layer forming the channel region may have an active polymer surrounding hydrophilic nano particles by filling a space therebetween or a hydrophilic nano particle layer formed on an active polymer layer depending on coated order of the hydrophilic nano particle and the active polymer.

Another aspect of the present invention provides a method of fabricating a biosensor having a transistor structure, including: forming a gate electrode on a substrate; forming a gate insulating layer on the gate electrode; forming source and drain electrodes on the gate insulating layer; and forming an active layer of an active polymer sensing an antigen-antibody reaction and a hydrophilic nano particle through direct printing in a channel region formed between the source and drain electrodes.

The forming of the active layer in the channel region may be performed by a first process including coating an ink containing a hydrophilic nano particle on a channel region and annealing the coated result, and after the annealing, coating an ink containing an active polymer and annealing the coated result, a second process including coating an ink containing an active polymer on the channel region and annealing the coated result, and after the annealing, coating an ink containing a hydrophilic nano particle and annealing the coated result, or a third process including coating an ink containing both the hydrophilic nano particle and the active polymer and annealing the coated result.

The ink containing the active polymer may be a solution formed by dispersing an active polymer having a conductive main chain and a side chain substituted with an aptamer or label in a solvent, and the ink containing the hydrophilic nano particle may be a solution formed by dispersing a nano particle, a cation interfacial active agent and a hydrophilic polymer in a solvent.

### BRIEF DESCRIPTION OF THE DRAWINGS

The above and other objects, features and advantages of the present invention will become more apparent to those of ordinary skill in the art by describing in detail preferred embodiments thereof with reference to the attached drawings in which:

FIG. 1 is a schematic cross-sectional view of a biosensor according to an exemplary embodiment of the present invention;

FIGS. 2A to 2C are views showing processes of fabricating and detecting a biosensor according to an exemplary embodiment of the present invention;

FIGS. 3A to 3C are views showing processes of fabricating and detecting a biosensor according to another exemplary embodiment of the present invention; and

FIGS. 4A and 4B are views showing processes of fabricating and detecting a biosensor according to still another exemplary embodiment of the present invention.

### DETAILED DESCRIPTION OF EXEMPLARY EMBODIMENTS

Hereinafter, the present invention will be described with reference to the accompanying drawings in detail. This invention may, however, be embodied in different forms and should not be construed as limited to the embodiments set forth herein. Rather, these embodiments are provided so that this disclosure will be thorough and complete, and will fully convey the scope of the invention to those skilled in the art. Like numbers refer to like elements throughout the specification. In the drawings, the thicknesses of layers and regions are exaggerated for clarity.

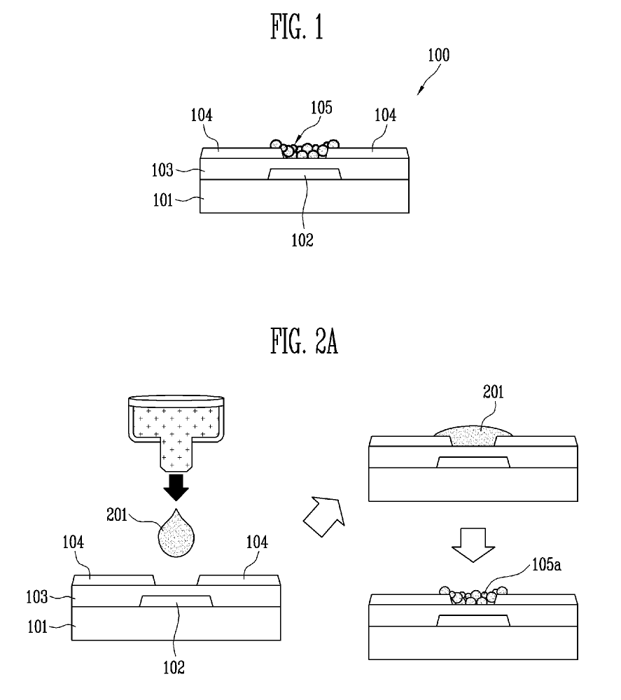

FIG. 1 is a schematic cross-sectional view of a biosensor according to an exemplary embodiment of the present invention.

Referring to FIG. 1, the biosensor 100 according to the present invention has a transistor structure including a gate electrode 102 formed on a substrate 101, a gate insulating layer 103 formed on the gate electrode, source and drain electrodes 104 formed on the gate insulating layer, and a channel region formed between the source and drain electrodes. The channel region includes an active layer 105, which is formed of an active polymer sensing an antigen-antibody reaction and a hydrophilic nano particle.

The substrate 101 may be formed of a material generally used in the art, or plastic.

The gate electrode 102, the gate insulting layer 103 and the source and drain electrodes 104 formed on the substrate 101 may be formed by a conventional method using materials generally used in an organic thin film transistor field, and then patterned. The source and drain electrodes may be formed in parallel or engaged with each other.

The active layer 105 is formed in the channel region between the source and drain electrodes 104 using an active polymer having a conductive main chain showing semiconductor characteristics and a side chain substituted with an aptamer or a label specifically binding to a target material to be detected and a hydrophilic nano particle capable of increasing hydrophilicity and reactivity of the polymer.

Here, examples of the conductive main chains showing semiconductor characteristics include, but are not limited to, poly(3-hexylthiophene) (P3HT), poly(9,9-dioctylfluorene-co-bithiophene) (F8T2) or poly(3,3′-didodecyl-quaterthiophene) (PQT-12). The aptamer or label substituting for the side chain may be dependant on the target material, which may be biotin.

The hydrophilic nano particle may include a nano particle such as alumina (Al₂O₃), aluminum nitride (AlN), silicon oxide (SiO₂) or boron oxide (B₂O₃), but the present invention is not limited thereto. To give hydrophilicity to the nano particle, a combination of a cation interfacial active agent such as tetramethyl ammonium methylchloride and a hydrophilic polymer such as butadiene and styrene may be used.

The biosensor having this structure easily detects or identifies whether a corresponding target material is present or not by an electrical change of the channel region of the transistor, which occurs when exposed to the target material using an active polymer having a side chain substituted with an aptamer or label specifically binding to the target material, for example, protein, peptide, amino acid or an organic or inorganic compound, and a conductive main chain showing semiconductor characteristics.

According to this principle, when a voltage is not applied to the gate, the biosensor may be used as a resistor which measures current between the source and drain while applying a voltage to the source and drain electrodes.

Hereinafter, a method of fabricating a biosensor according to the present invention will be described in further detail with reference to exemplary embodiments.

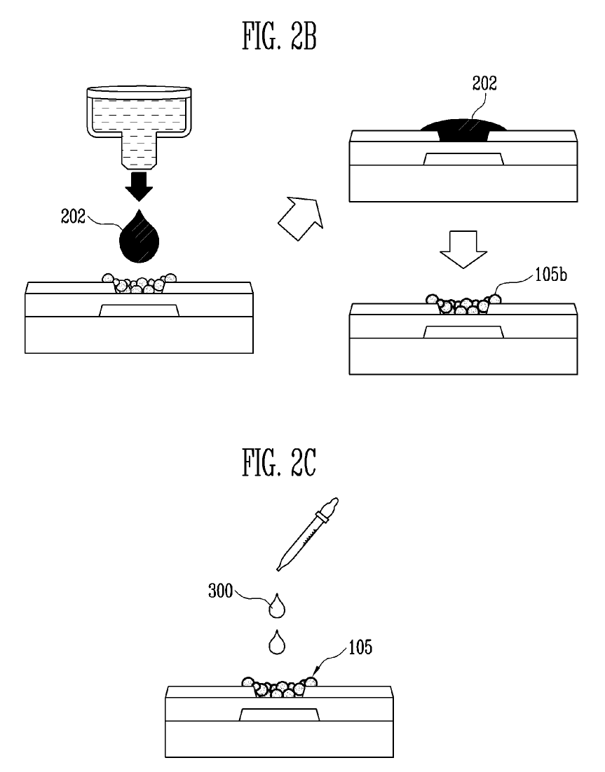

FIGS. 2A to 2C are views showing processes of fabricating and detecting a biosensor according to an exemplary embodiment of the present invention.

Referring to FIG. 2A, a gate electrode 102, a gate insulating layer 103 and source and drain electrodes 104 are sequentially stacked on a substrate 101. An ink 201 containing a hydrophilic nano particle is coated on a channel region disposed between the source and drain electrodes through direct printing, and annealed at proper temperature and for a proper period of time to evaporate a solvent, thereby finally forming a hydrophilic nano particle layer 105a.

As shown in FIG. 2B, an ink 202 containing an active polymer material is coated on the hydrophilic nano particle layer 105a, and then annealed at proper temperature and for a proper period of time to evaporate a solvent, thereby finally forming an active polymer layer 105b sensing an antigen-antibody reaction. Thus, the biosensor 100 having a transistor structure is completed.

As shown in FIG. 2C, in the biosensor 100, a solution 300 containing a specific molecule is sprayed on an active layer 105 in which the hydrophilic nano particle layer 105a is surrounded by the active polymer layer 105b to detect and identify a corresponding specific molecule by an electrical change of the channel region.

An example of the direct printing is inkjet printing which can print a pattern on the substrate formed of various materials since an ink is sprayed to a target position in a non-contact manner. Thus, the substrate to which inkjet printing will be applied needs to be subjected to surface treatment to form a three dimensional structure after a droplet of the ink is dried. Generally, to increase a contact angle of the ink, the substrate needs to be subjected to hydrophobic treatment. Since the ink 202 containing the active polymer also has hydrophobic characteristics, when the solution 300 containing a specific material is sprayed to the active polymer layer to bind an antigen with an antibody, hydrophilicity and reactivity are degraded, thereby deteriorating the biosensor's performance. To overcome this problem, before coating the active polymer layer 105b, the hydrophilic nano particle layer 105a may be coated using the ink 201 containing a hydrophilic nano particle by inkjet printing. However, a similar effect can be obtained by coating the active polymer and then coating the hydrophilic nano particle or by a mixture of the active polymer and the hydrophilic nano particle.

The hydrophilic ink 201 containing a nano particle to form the hydrophilic nano particle layer 105a is composed of a nano particle such as alumina (Al₂O₃), aluminum nitride (AlN), silicon oxide (SiO₂) or boron oxide (B₂O₃), a cation interfacial active agent such as tetramethyl ammonium chloride (TMAC), and a hydrophilic polymer such as butadiene or styrene. A solvent may be methanol, isopropanol, chloroform or tetrahydrofuran (THF).

The nano particle, the cation interfacial active agent and the hydrophilic polymer may be mixed with a solvent to have a proper viscosity of 10 to 30 cps for inkjet printing. The hydrophilic nano particle layer having this composition functions for the biosensor to obtain good hydrophilicity and reaction results by easily absorbing a solution 300 having a target material.

The ink 202 containing the active polymer is formed by dispersing an active polymer having a conductive main chain of poly(3-hexylthiopene) (P3HT), poly(9,9-dioctylfluorene-co-bithiophene) (F8T2) or poly(3,3′-didodecyl-quaterthiophene) (PQT-12) and a side chain substituted with an aptamer or label in a solvent such as methanol, isopropanol, chloroform or THF to have a viscosity of 10 to 30 cps, which is suitable for inkjet printing.

An ink 203 containing the hydrophilic nano particle and the active polymer is formed by dispersing a hydrophilic nano particle and an active polymer in a solvent such as methanol, isopropanol, chloroform or THF to have a viscosity of 10 to 30 cps, which is suitable for inkjet printing.

Annealing to remove the solvent used when the hydrophilic nano particle layer 105a and the active polymer layer 105b are formed may be performed at various temperatures, which are dependant upon the kind of the solvent used, and preferably 100 to 200° C. for 10 minutes to 1 hour.

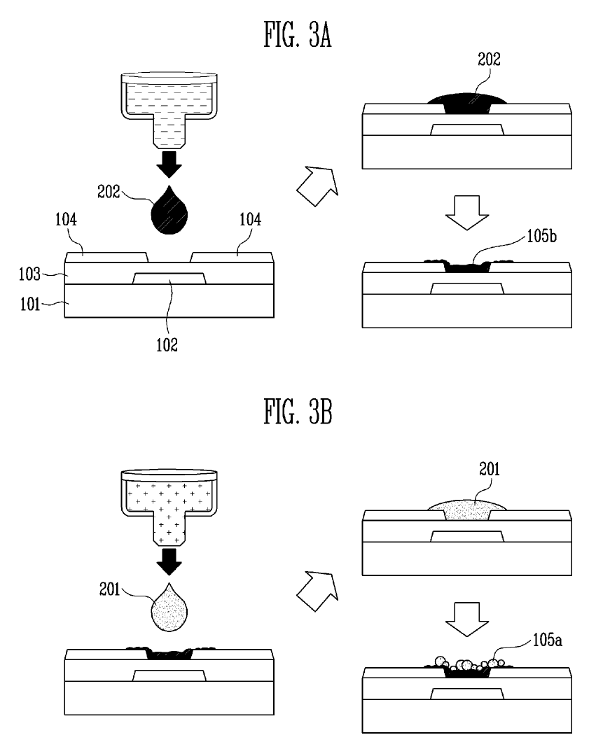

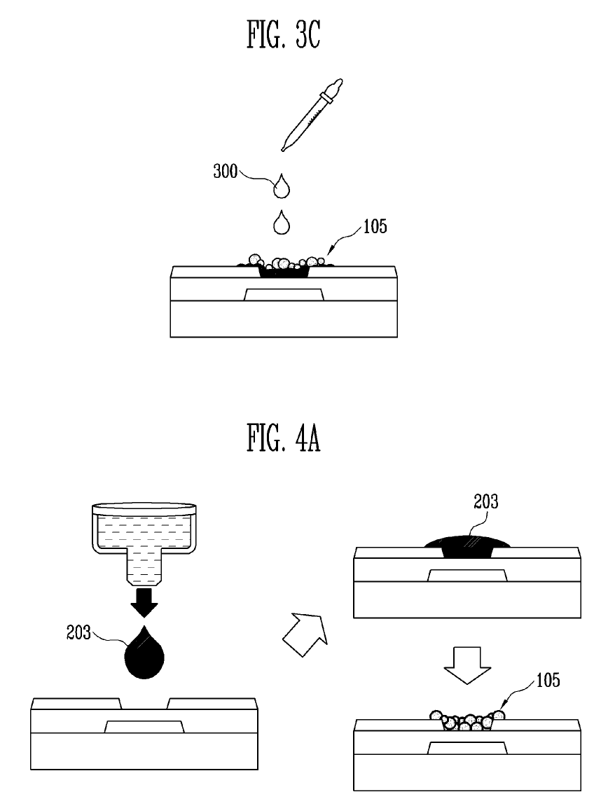

FIGS. 3A to 3C are views showing processes of fabricating and detecting a biosensor according to another exemplary embodiment of the present invention.

Referring to FIG. 3A, a gate electrode 102, a gate insulating layer 103 and source and drain electrodes 104 are sequentially stacked on a substrate 101. An ink 202 containing an active polymer is coated on a channel region disposed between the source and drain electrodes through direct printing, and annealed at proper temperature and for a proper period of time to evaporate a solvent, thereby finally forming an active polymer layer 105b.

Subsequently, as shown in FIG. 3B, an ink 201 containing a hydrophilic nano particle is coated on the active polymer layer 105b, and annealed at proper temperature and for a proper period of time to remove a solvent, thereby forming a hydrophilic nano particle layer 105a. Thus, a biosensor 100 having a transistor structure is completed.

As shown in FIG. 3C, a solution 300 containing a specific molecule is sprayed to an active layer 105 having the hydrophilic nano particle layer 105a formed on the active polymer layer 105b of the biosensor 100 to detect and identify a corresponding specific molecule by an electric change of the channel region.

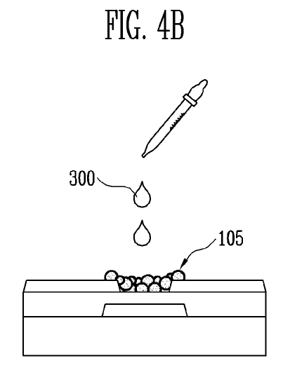

FIGS. 4A and 4B are views showing processes of fabricating and detecting a biosensor according to still another exemplary embodiment of the present invention.

Referring to FIG. 4A, a gate electrode 102, a gate insulating layer 103 and source and drain electrodes 104 are sequentially stacked on a substrate 101. An ink 203 containing an active polymer material and a hydrophilic nano particle is coated on a channel region disposed between the source and drain electrodes through direct printing, and annealed at proper temperature and for a proper period of time to evaporate a solvent, thereby finally forming an active layer 105.

Subsequently, as shown in FIG. 4B, a solution 300 having a specific molecule is sprayed to the active layer 105 of a biosensor 100 to detect and identify a corresponding specific molecule by an electric change of the channel region.

As described above, a biosensor having a transistor structure is fabricated using an active polymer sensing an antigen-antibody reaction and a hydrophilic nano particle through direct printing, and has the following effects:

First, due to increases in hydrophilicity and reactivity between an antigen and an antibody, sensitivity and selectivity can be improved;

Second, compared to a conventional biosensor based on an inorganic material such as Si, it is not necessary to perform immobilization to provide fixation to a surface of an inorganic material; and

Third, a large-area process can be possible through direct printing, and a device can be easily fabricated on various substrates formed of, for example, plastic.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A biosensor having a transistor structure, comprising:
a gate electrode formed on a substrate;
a gate insulating layer formed on the gate electrode;
source and drain electrodes formed on the gate insulating layer; and
a channel region formed between the source and drain electrodes,
wherein the channel region includes an active layer formed of an active polymer sensing an antigen-antibody reaction and a hydrophilic nano particle.

2. The biosensor according to claim 1, wherein the active polymer sensing the antigen-antibody reaction has a conductive main chain showing semiconductor characteristics, and a side chain substituted with an aptamer or label specifically binding to a target material.

3. The biosensor according to claim 2, wherein the conductive main chain of the active polymer is poly(3-hexylthiophene) (P3HT), poly(9,9-dioctylfluorene-co-bithiophene) (F8T2) or poly(3,3′-didodecyl-quaterthiophene) (PQT-12), and the side chain is biotin.

4. The biosensor according to claim 1, wherein the hydrophilic nano particle is a composition of a nano particle such as alumina (Al₂O₃), aluminum nitride (AlN), silicon oxide (SiO₂) or boron oxide (B₂O₃), a cation interfacial active agent and a hydrophilic polymer.

5. The biosensor according to claim 1, wherein the active layer has an active polymer surrounding hydrophilic nano particles by filling a space therebetween.

6. The biosensor according to claim 1, wherein the active layer has a hydrophilic nano particle layer formed on an active polymer layer.

7. A method of fabricating a biosensor having a transistor structure, comprising:
forming a gate electrode on a substrate;
forming a gate insulating layer on the gate electrode;
forming source and drain electrodes on the gate insulating layer; and
forming an active layer of an active polymer sensing an antigen-antibody reaction and a hydrophilic nano particle through direct printing in a channel region formed between the source and drain electrodes.

8. The method according to claim 7, wherein the forming of the active layer in the channel region comprises:
coating an ink containing a hydrophilic nano particle on the channel region and annealing the coated result; and
after the annealing, coating an ink containing an active polymer and annealing the coated result.

9. The method according to claim 7, wherein the forming of the active layer in the channel region comprises:
coating an ink containing an active polymer on the channel region and annealing the coated result; and
after the annealing, coating an ink containing a hydrophilic nano particle and annealing the coated result.

10. The method according to claim 7, wherein the forming of the active layer in the channel region comprises coating an ink containing both the hydrophilic nano particle and the active polymer, and annealing the coated result.

11. The method according to claim 8, wherein the ink containing the active polymer is a solution formed by dispersing an active polymer having a conductive main chain and a side chain substituted with an aptamer or label in a solvent.

12. The method according to claim 8, wherein the ink containing the hydrophilic nano particle is a solution formed by dispersing a nano particle, a cation interfacial active agent, and a hydrophilic polymer in a solvent.

13. The method according to claim 9, wherein the ink containing the active polymer is a solution formed by dispersing an active polymer having a conductive main chain and a side chain substituted with an aptamer or label in a solvent.

14. The method according to claim 9, wherein the ink containing the hydrophilic nano particle is a solution formed by dispersing a nano particle, a cation interfacial active agent, and a hydrophilic polymer in a solvent.

15. The method according to claim 10, wherein the ink containing the hydrophilic nano particle and the active polymer is a solution formed by dispersing a hydrophilic nano particle composed of a nano particle, a cation interfacial active agent and a hydrophilic polymer and an active polymer in a solvent.
